(19) United States
(12) Patent Application Publication
(10) Pub. No.: US 2024/0344068 A1
(43) Pub. Date: Oct. 17, 2024

(54) DUAL FUNCTION HYBRID NANOPARTICLES AND METHODS OF USING THE SAME TO TREAT DISEASES AND DISORDERS

(71) Applicant: UNIVERSITY OF SOUTH FLORIDA, Tampa, FL (US)

(72) Inventors: Oksana Fihurka, Wesley Chapel, FL (US); Vasyl Sava, Seffner, FL (US); Juan Sanchez-Ramos, Temple Terrace, FL (US)

(21) Appl. No.: 18/622,278

(22) Filed: Mar. 29, 2024

Related U.S. Application Data
(60) Provisional application No. 63/493,569, filed on Mar. 31, 2023.

(51) Int. Cl. C12N 15/113; A61K 9/127; A61K 31/00; A61K 47/36; A61P 25/28

(52) U.S. Cl. CPC C12N 15/113; A61K 9/127; A61K 9/1278; A61K 31/658; A61K 47/36; A61P 25/28; C12N 2310/14; C12N 2310/315; C12N 2310/321

(57) ABSTRACT

Disclosed herein are siRNAs, hybrid nanoparticles comprising the siRNAs, pharmaceutical compositions comprising the same, and methods of making and using the same to treat neurological diseases and disorders.

### CROSS-REFERENCE TO RELATED APPLICATIONS

The present application claims priority to U.S. Provisional Patent Application No. 63/493,569 that was filed Mar. 31, 2023, the entire contents of which are hereby incorporated by reference.

### STATEMENT REGARDING FEDERALLY SPONSORED RESEARCH OR DEVELOPMENT

This invention was made with government support under NS095563 awarded by The National Institutes of Health. The government has certain rights in the invention.

### SEQUENCE LISTING

A Sequence Listing accompanies this application and is submitted as an xml file of the sequence listing named “173738_02740.xml” which is 6,199 bytes in size and was created on Mar. 12, 2024. The sequence listing is electronically submitted via Patent Center and is incorporated herein by reference in its entirety.

### BACKGROUND

Advances in nanotechnology have contributed to the development of novel nanocarriers for gene therapy of human hereditary diseases. Huntington's disease (HD) is caused by a mutation in the huntingtin (HTT) gene in which the same trinucleotide (CAG) is abnormally repeated. Gene silencing mediated by short interfering RNA (“siRNA”) can be used to treat HD by downregulation of mutant HTT expression. Naked siRNA is negatively charged, which hinders its cellular internalization, and it therefore needs a protective carrier. Development of the appropriate protective carrier and effective routes of administration is critical for successful gene therapy. Accordingly, novel delivery strategies for siRNA are needed in the art, especially with regard to strategies to treat Huntington's disease.

### SUMMARY

In an aspect of the current disclosure, nanoparticles are provided. In some embodiments, the nanoparticles comprise: (i) a core comprising at least one siRNA; and (ii) a phospholipid outer layer. In some embodiments, the core comprises or further comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-dioleoyl-sn-glycero-3-phosphocholine (DOPC), or both DPPC and DOPC. In some embodiments, the phospholipid outer layer comprises DPPC, DOPC, and cannabidiol (CBD).

In another aspect of the current disclosure, further nanoparticles are provided. In some embodiments, the nanoparticles comprise: (i) a core comprising at least one siRNA; and (ii) a phospholipid outer layer; wherein the core comprises chitosan lactate and the phospholipid outer layer comprises DPPC, DOPC, and cannabidiol (CBD). In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the Zeta Potential (z-potential or (potential) of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter.

In another aspect of the current disclosure, siRNAs are provided. In some embodiments, the siRNAs comprise SEQ ID NO: 1, or a sequence at least 90% similar to SEQ ID NO: 1. In some embodiments, the siRNA comprises at least one modified nucleotide. In some embodiments, the at least one modified nucleotide comprises an O-methylated nucleotide or a phosphorothioate-modified nucleotide.

In another aspect of the current disclosure, pharmaceutical compositions are provided. In some embodiments, the pharmaceutical compositions comprise a nanoparticle comprising: (i) a core comprising at least one siRNA; and (ii) a phospholipid outer layer. In some embodiments, the core comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-diolcoyl-sn-glycero-3-phosphocholine (DOPC), or both DPPC and DOPC. In some embodiments, the phospholipid outer layer comprises DPPC, DOPC, and cannabidiol (CBD). In some embodiments, the pharmaceutical compositions comprise at least one pharmaceutically acceptable carrier or excipient.

In some embodiments, the pharmaceutical compositions comprise a nanoparticle comprising: (i) a core comprising at least one siRNA; and (ii) a phospholipid outer layer; wherein the core comprises chitosan lactate and the phospholipid outer layer comprises DPPC, DOPC, and cannabidiol (CBD). In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the z-potential of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter. In some embodiments, the pharmaceutical compositions comprise at least one pharmaceutically acceptable carrier or excipient.

In another aspect of the current disclosure, further pharmaceutical compositions are provided. In some embodiments, the pharmaceutical compositions comprise an siRNA comprising SEQ ID NO: 1, or a sequence at least 90% similar to SEQ ID NO: 1. In some embodiments, the siRNA comprises at least one modified nucleotide. In some embodiments, the at least one modified nucleotide comprises an O-methylated nucleotide or a phosphorothioate-modified nucleotide. In some embodiments, the pharmaceutical compositions comprise at least one pharmaceutically acceptable carrier or excipient.

In another aspect of the current disclosure, methods are provided. In some embodiments, the methods comprise administering a pharmaceutical composition comprising (i) a core comprising at least one siRNA; and (ii) a phospholipid outer layer. In some embodiments, the core comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-dioleoyl-sn-glycero-3-phosphocholine (DOPC), or both DPPC and DOPC. In some embodiments, the phospholipid outer layer comprises DPPC, DOPC, and cannabidiol (CBD). In some embodiments, the pharmaceutical compositions comprise at least one pharmaceutically acceptable carrier or excipient. In some embodiments, the subject is suffering from a neurological disease or disorder. In some embodiments, administration comprises intranasal administration.

In some embodiments the methods comprise administering a pharmaceutical composition comprising: (i) a core comprising at least one siRNA; and (ii) a phospholipid outer layer; wherein the core comprises chitosan lactate and the phospholipid outer layer comprises DPPC, DOPC, and cannabidiol (CBD). In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the z-potential of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter. In some embodiments, the pharmaceutical compositions comprise at least one pharmaceutically acceptable carrier or excipient. In some embodiments, the subject is suffering from a neurological disease or disorder. In some embodiments, administration comprises intranasal administration.

In another aspect of the current disclosure, methods of treating a neurological disease or disorder in a subject in need thereof are provided. In some embodiments, the method comprises administering a therapeutically effective amount of a pharmaceutical composition comprising: (i) a core comprising at least one siRNA; and (ii) a phospholipid outer layer to a subject to treat the neurological disease or disorder. In some embodiments, the core comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-diolcoyl-sn-glycero-3-phosphocholine (DOPC), or both DPPC and DOPC. In some embodiments, the phospholipid outer layer comprises DPPC, DOPC, and cannabidiol (CBD). In some embodiments, the pharmaceutical compositions comprise at least one pharmaceutically acceptable carrier or excipient. In some embodiments, the method reduces inflammation in the subject. In some embodiments, administration comprises intranasal administration.

In some embodiments, the method comprises administering a therapeutically effective amount of a pharmaceutical composition comprising: (i) a core comprising at least one siRNA; and (ii) a phospholipid outer layer; wherein the core comprises chitosan lactate and the phospholipid outer layer comprises DPPC, DOPC, and cannabidiol (CBD) to a subject to treat the neurological disease or disorder. In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the z-potential of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter. In some embodiments, the pharmaceutical compositions comprise at least one pharmaceutically acceptable carrier or excipient. In some embodiments, the method reduces inflammation in the subject. In some embodiments, administration comprises intranasal administration.

In another aspect of the current disclosure, methods of reducing expression of a gene related to a neurological disease or disorder and/or reducing inflammation in a subject in need thereof, are provided. In some embodiments, the methods comprise administering a pharmaceutical composition comprising: (i) a core comprising at least one siRNA; and (ii) a phospholipid outer layer; to a subject to reduce expression of a gene related to a neurodegenerative disease and reduce inflammation or neuroinflammation in the subject. In some embodiments, the core comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-diolcoyl-sn-glycero-3-phosphocholine (DOPC), or both DPPC and DOPC. In some embodiments, the phospholipid outer layer comprises DPPC, DOPC, and cannabidiol (CBD). In some embodiments, the pharmaceutical compositions comprise at least one pharmaceutically acceptable carrier or excipient. In some embodiments, the neurological disease or disorder is a neurodegenerative disease. In some embodiments, the neurodegenerative disease is selected from the group consisting of Huntington's disease, familial Parkinson's disease, familial Alzheimer's disease, and familial amyotrophic lateral sclerosis. In some embodiments, the neurodegenerative disease is Huntington's disease. In some embodiments, the subject is suffering from Huntington's disease and the pharmaceutical composition comprises a nanoparticle comprising a core comprising at least one siRNA comprising SEQ ID NO: 1. In some embodiments, the lipid outer layer comprises cannabidiol. In some embodiments, the method reduces IL-6 levels in the subject. In some embodiments, administration comprises intranasal administration.

In some embodiments, the methods comprise administering a pharmaceutical composition comprising: i) a core comprising at least one siRNA; and (ii) a phospholipid outer layer; wherein the core comprises chitosan lactate and the phospholipid outer layer comprises DPPC, DOPC, and cannabidiol (CBD) to a subject to reduce expression of a gene related to a neurodegenerative disease and reduce inflammation or neuroinflammation in the subject. In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the z-potential of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter. In some embodiments, the pharmaceutical compositions comprise at least one pharmaceutically acceptable carrier or excipient. In some embodiments, the method reduces inflammation in the subject. In some embodiments, the neurological disease or disorder is a neurodegenerative disease. In some embodiments, the neurodegenerative disease is selected from the group consisting of Huntington's disease, familial Parkinson's disease, familial Alzheimer's disease, and familial amyotrophic lateral sclerosis. In some embodiments, the neurodegenerative disease is Huntington's disease. In some embodiments, the subject is suffering from Huntington's disease and the pharmaceutical composition comprises a nanoparticle comprising a core comprising at least one siRNA comprising SEQ ID NO: 1. In some embodiments, the lipid outer layer comprises cannabidiol. In some embodiments, the method reduces IL-6 levels in the subject. In some embodiments, administration comprises intranasal administration.

In another aspect of the current disclosure, methods of making a nanoparticle are provided. In some embodiments, the nanoparticle comprises: (i) a core comprising at least one siRNA; and (ii) a phospholipid outer layer. In some embodiments, the core comprises chitosan or chitosan lactate. In some embodiments, the core comprises chitosan, chitosan lactate, or a combination of chitosan and chitosan lactate. In some embodiments, the core comprises chitosan lactate. In some embodiments, the phospholipid outer layer comprises at least one anti-inflammatory compound. In some embodiments, the at least one anti-inflammatory compound comprises cannabidiol (CBD). In some embodiments, the at least one siRNA comprises SEQ ID NO: 1. In some embodiments, the phospholipid outer layer comprises dipalmitoylphosphatidylcholine (DPPC), 1,2-diolcoyl-sn-glycero-3-phosphocholine (DOPC), or both DPPC and DOPC. In some embodiments, the phospholipid outer layer comprises DPPC, DOPC, and cannabidiol (CBD).

In some embodiments the nanoparticle comprises: i) a core comprising at least one siRNA; and (ii) a phospholipid outer layer; wherein the core comprises chitosan lactate and the phospholipid outer layer comprises DPPC, DOPC, and cannabidiol (CBD). In some embodiments, the at least one siRNA comprises SEQ ID NO: 1. In some embodiments, the final ratio of CS and/or CSL:DPPC:DOPC:CBD is about 20:12:10.5:7.5:1. In some embodiments, the z-potential of the nanoparticle is about 6 mV to about 55 mV. In some embodiments, the z-potential of the nanoparticle is about 6 mV to about 8 mV. In some embodiments, the nanoparticle is about 150 to about 210 nm in diameter. In some embodiments, the nanoparticle is about 195 to about 207 nm in diameter.

### BRIEF DESCRIPTION OF THE FIGURES

The patent or application file contains at least one drawing executed in color. Copies of this patent or patent application publication with color drawing(s) will be provided by the Office upon request and payment of the necessary fec.

FIG. 1. Efficiency of polymeric nanoparticle and liposome fusion into hybrid nanostructures. Two-way analysis of variance followed by multiple comparisons test revealed that both the dye variables (Hoechst vs Nile red) and the nanostructure variables contributed significantly to total variance; it also revealed a significant interaction between variables. Hoechst dye was significantly reduced in hybrid nanoparticles compared with nanoparticles. Data are expressed as mean standard deviation (n=3). * p<0.05. NP: Nanoparticle.

FIG. 2. Cytotoxicity of chitosan- and chitosan lactate-based nanoparticles and hybrid nanoparticles in the bone marrow mesenchymal stem cell line. Data are expressed as mean ±standard deviation percentage of dead cells among alive cells (n=4). Standard deviation is denoted by error bars. CS- and CSL-based NPs and HNPs were compared using two-way analysis of variance multiple comparisons test. ** p<0.01; *** p<0.001. ANOVA: Analysis of variance; CS: Chitosan; CSL: Chitosan lactate; HNP: Hybrid nanoparticle; NP: Nanoparticle; pDNA: Plasmid DNA; SD: Standard deviation.

FIG. 3. Comparative transfection efficacy of chitosan and chitosan lactate nanoparticles and hybrid nanoparticles. (A) Kinetic curves obtained by extrapolation experimental data (Table 2) with proposed logistic function. Fitting parameters R and the correspondent Pearson's coefficients (P) for the experimental and calculated data were as follows: R=1.5 and P=0.998 for the CSL NP; R=1.1 and P=0.995 for the CSL HNP; R=1.05 and P=0.997 for the CS NP; R=0.89 and P=0.997 for the CS HNP. (B) 50% transfection level for pDNA carried by different nanocarriers in BMMS cell culture. CS: Chitosan; CSL: Chitosan lactate; HNP: Hybrid nanoparticle; NP: Nanoparticle; pDNA Plasmid DN4.

FIG. 4. In vitro Huntington's disease gene silencing efficacy of two anti-HTT siRNAs and a no template control loaded into commercially available Lipofectamine 3000. BMMS cells were plated for 48 h before treatment with siRNA for 24 h in OptiMEM® medium. The graph represents changes in mutant HTT gene expression RQ-1. Data are expressed as mean standard error of the mean of triplicates. Statistical analysis was conducted using one-way ANOVA followed by Tukey post hoc multiple comparisons test. Both siRNA preparations significantly reduced HTT expression compared with NTC. * p<0.001. ANOVA: Analysis of variance; BMMS: Bone marrow mesenchymal stem; NTC: No template control.

FIG. 5. In vitro gene silencing efficacy of anti-HTT siRNAs packaged into chitosan lactate-based nanoparticles and hybrid nanoparticles. BMMS cells were plated for 48 h before treatment with siRNAs containing NPs for 24 h in OptiMEM® medium. The graph represents changes in human HTT gene expression RQ-1. Data are expressed as mean standard error of the mean (n=4). Statistical analysis was conducted using one-way analysis of variance followed by Tukey post hoc multiple comparisons test. ns=p>0.05. * p<0.05. BMMS: Bone marrow mesenchymal stem; CSL: Chitosan lactate; HNP: Hybrid nanoparticle; NS: Not significant; NP: Nanoparticle.

FIG. 6. In vitro evaluation of lipopolysaccharide-stimulated IL-6 expression in bone marrow mesenchymal stem cells treated with 1 μg/ml lipopolysaccharide or 1 μg/ml lipopolysaccharide in combination with chitosan lactate-based hybrid nanoparticles or chitosan lactate-based hybrid nanoparticles loaded with cannabidiol. Data are expressed as mean ±standard deviation of three independent experiments. Statistical analysis was conducted using one-way analysis of variance followed by Tukey post hoc multiple comparisons test. * p<0.05; *** p<0.001. CBD: Cannabidiol; HNP: Hybrid nanoparticle; LPS: Lipopolysaccharide; SD: Standard deviation; w/o: Without.

FIGS. 7A, 7B, 7C, and 7D. Uptake of nanoparticles packed with Cy3-labeled siRNA in bone marrow mesenchymal stem cells. Cells were plated 3 days before siRNA delivery. HNPs with Cy3-labeled siRNA were added to the cells for 6 h. After 6 h, the cells were rinsed twice with PBS and immediately observed using confocal microscopy. Representative images of CSL-based HNP-treated cells are shown at 10× magnification. Cells were stained with (A) calcein or (B) Hoechst 33342. (C) HNPs loaded with Cy3-labeled siRNA. (D) Overlay of the three panels with intracellular yellow signal indicating Cy3-labeled siRNA within calcein-positive cells. Scale bar=100 nm. CSL: Chitosan lactate; HNP: Hybrid nanoparticle; PBS: Phosphate-buffered saline.

FIG. 8. Shows a graphical representation of one embodiment of the disclosed nanoparticles.

### DETAILED DESCRIPTION

Nanoparticles (NPs) composed of polycations demonstrate the ability to protect genetic material from degradation and facilitate cellular entry. Intranasal administration of chitosan (CS)-based NPs carrying siRNA has been reported to significantly lower gene expression. Unlike existing gene therapies for brain disease that require intrathecal or direct intracerebral injection, approaches leveraging nanoparticles for drug delivery intranasally are noninvasive, do not rely on viral vectors, and allow for safe chronic intermittent delivery of gene silencing agents. Furthermore, novel treatments for the chronic neurodegenerative process triggered by accumulation of mutant huntingtin (HTT) protein, i.e., Huntington's disease, are sorely needed to combat this debilitating and fatal disease. Going forward, gene therapies will need to be developed that both silence the gene and suppress neuroinflammatory processes.

Genetic disorders can cause temporary or permanent neural damage, but studies have shown that cannabidiol (CBD) is able to protect against this damage and improve recovery. CBD, as a neuroprotectant, helps reduce damage to the brain and nervous system and encourages the growth and development of new neurons. The possible therapeutic application of cannabinoids as a result of these anti-inflammatory properties has been tested in a variety of preclinical models. In spite of potency and a variety of favorable pharmacological effects, the clinical use of CBD is limited because of reduced stability and poor solubility in water.

Liposomes are considered important candidates for the improvement of drug-delivery systems, especially with regard to the simultaneous delivery of hydrophilic and hydrophobic drugs. The use of a lipid shell to surround an NP loaded with the payload, e.g., siRNA, can be beneficial because it allows control of surface charge, further enhancing cargo protection by changing stability and lipophilicity. Even though codelivery of multiple therapeutics is challenging, this approach may lead to greater therapeutic benefits. Hybrid NPs (HNPs) are liposomal structures in which NPs are encapsulated in the aqueous core of the liposome and a lipophilic therapeutic agent is embedded in the lipid layer. HNPs are a versatile platform of multifunctional particles combining the advantageous properties of liposomes and NPs to encapsulate, transport, deliver, protect, and prolong the biological half-life of hydrophobic, e.g., CBD, and hydrophilic, e.g., siRNA, therapeutics.

A novel efficient siRNA is disclosed that specifically targets the mutant HTT gene using bioinformatics tools, web databases open to the public, and guidelines for siRNA design incorporating both the structural features of the targeted RNAs and the sequence features of the siRNAs. Furthermore, the inventors have established mesenchymal stem cell lines essential for modeling HD in cells. The genetically engineered FVB-Tg YAC128/53Hay/J mouse line (expressing a full-length human mutant HTT gene) serves as a mouse model of HD. The cell line derived from bone marrow mesenchymal stem (BMMS) cells expresses mutant HTT and was used in the studies reported here of gene silencing and anti-inflammatory action at the cellular level.

Multifunctional HNP delivery systems have been employed recently in cancer therapy and immunotherapy as an approach to enable medical visualization. The disclosed concept of application of HNPs offers a new direction in gene therapy. Hybrid nanocarriers are presently disclosed with dual payload with the ability to lower HTT gene expression and/or attenuate inflammation. The combination of cannabinoid and siRNA in an all-in-one system provides a novel, and non-obvious, useful multimodal approach for gene silencing and concomitant reduction of inflammation.

### SiRNA

The disclosed compositions (double-stranded RNAs (dsRNAs), small interfering RNAs (siRNAs)) function through a process known as RNA interference (RNAi). The mechanism of action of siRNA is understood by the skilled person. Interfering RNA generally refers to a single-stranded RNA (ssRNA) or double-stranded RNA (dsRNA). After processing in a cell, dsRNA is capable of targeting specific messenger RNA (mRNA) and silencing (i.e., substantially or entirely inhibiting) the expression of a target gene. During this process, dsRNA (which may include shRNA) is enzymatically processed into short-interfering RNA (siRNA) duplexes of about 20-23 nucleotides in length. The anti-sense strand of the siRNA duplex is then incorporated into a cytoplasmic complex of proteins (RNA-induced silencing complex or RISC). The RISC complex containing the anti-sense siRNA strand also binds mRNA which has a sequence complementary to the anti-sense strand allowing complementary base-pairing between the anti-sense siRNA strand and the sense mRNA molecule. The mRNA molecule is then specifically cleaved by an enzyme (RNase) associated with RISC resulting in specific gene silencing. For gene silencing or knock down (i.e., mRNA cleavage) to occur, anti-sense RNA (i.e., siRNA) must become incorporated into the RISC. This represents an efficient process that occurs in nucleated cells during regulation of gene expression.

As such, siRNA-mediated RNA interference may be considered to involve two-steps: (i) an initiation step, and (ii) an effector step. In the first step, input siRNA is processed into small fragments, such as^{˜}21-23-nucleotide “guide sequences.” The guide sequences can be incorporated into the protein-RNA RISC complex which is capable of degrading mRNA. As such, the RISC complex acts in the second effector step to destroy mRNAs that are recognized by the guide RNAs through base-pairing interactions. RNA interference via use of siRNA may be considered to involve the introduction by any means of double stranded RNA into a cell which triggers events that cause the degradation of a target RNA, and as such siRNA may be considered to be a form of post-transcriptional gene silencing. The skilled person understands how to prepare and utilize siRNA molecules. (Sec, e.g., Hammond et al., Nature Rev Gen 2:110-119 (2001); and Sharp, Genes Dev 15:485-490 (2001), the contents of which are incorporated herein by reference in their entireties).

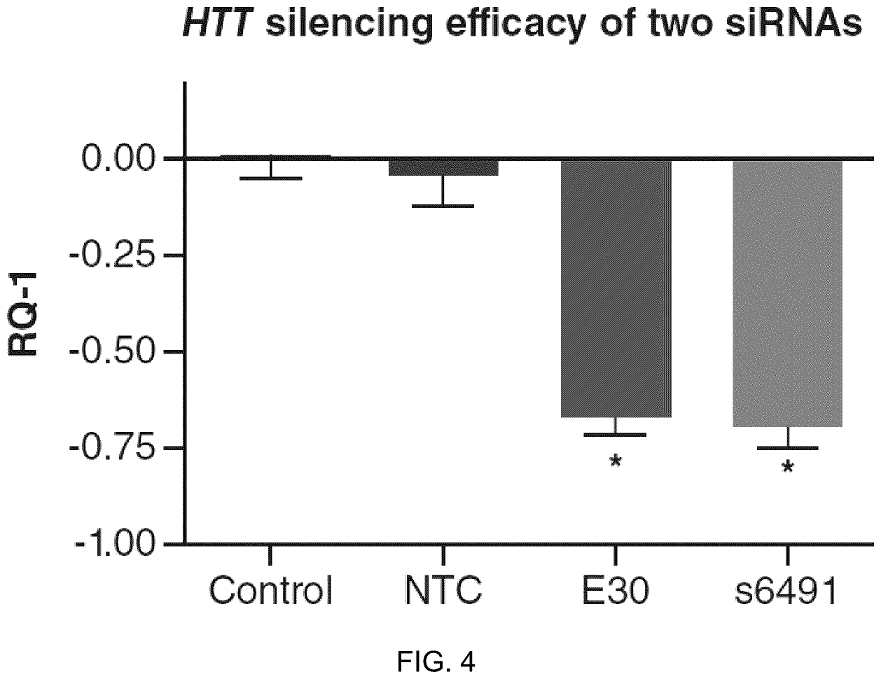

Novel siRNAs, e.g., (SEQ ID NO: 1) are presently disclosed, targeting huntingtin (HTT) which, when mutated, is the causative agent of Huntington's disease. Furthermore, the novel siRNA (E30, SEQ ID NO: 1) demonstrates effective knockdown of HTT in an in vitro model (FIG. 4).

As used herein, “siRNA” refers to a double-stranded RNA (dsRNA) of about 20 to about 25 nucleotides in length. The disclosed siRNAs may comprise nucleotide overhangs, modified nucleotides, or both nucleotide overhangs and modified nucleotides.

As used herein, “modified nucleotides” refer to nucleotide bases which are not found in nature and siRNAs comprising modified nucleotides may also be referred to as “modified siRNAs.” Exemplary modified nucleotides may include, but are not limited to, modified nucleotides such as 2′-O-methyl (2′OMe) nucleotides, 2′-deoxy-2′-fluoro (2′F) nucleotides, 2′-deoxy nucleotides, 2′-O-(2-methoxyethyl) (MOE) nucleotides, and the like. The preparation of modified siRNA is known by one skilled in the art. In some embodiments, the disclosed dsRNA molecules include one or more modified nucleotides at the 5′-terminus of the passenger strand of the dsRNA that prevent incorporation of the passenger strand into RISC. (See, e.g., Walton et al., Minireview: “Designing highly active siRNAs for therapeutic applications,” the FEBS Journal, 277 (2010) 4806-4813). Further, the disclosed compositions may comprise nucleotides with phosphorothioate modifications to the ribose sugar moiety on a nucleotide or “locked nucleic acids”, a type of modified nucleotide, which, as used herein, refers to nucleotides comprising a 2′-O, 4′-C methylene bridge.

The disclosed siRNAs may further comprise an “overhang” on one or both ends of the dsRNA. As used herein, “overhang” or “nucleotide overhang” refers to one or more nucleotides that extend beyond the dsRNA duplex on either the 5′ or 3′ end of one or both strands of the duplex. For example, SEQ ID NO: 2 comprises the sense strand of the siRNA (dsRNA) of SEQ ID NO: 1, with the addition of a 3′ nucleotide overhang, i.e., a UU overhang. SEQ ID NO: 3 comprises the anti-sense strand of SEQ ID NO: 1, targeting the HTT mRNA, but with the addition of a 3′ overhang, i.e., a GA overhang. Overhangs may comprise deoxyribonucleotide overhangs.

### Nanoparticles

Designed and selected HNPs are disclosed capable of carrying CBD and siRNA. These “hybrid nanocarriers” were optimized to maintain small particle size, high siRNA and CBD encapsulation, and effective gene downregulation efficacy, while minimizing cytotoxicity.

Accordingly, in an aspect of the current disclosure, nanoparticles are provided. In some embodiments, the nanoparticles comprise (i) a core comprising at least one siRNA; and (ii) a phospholipid outer layer.

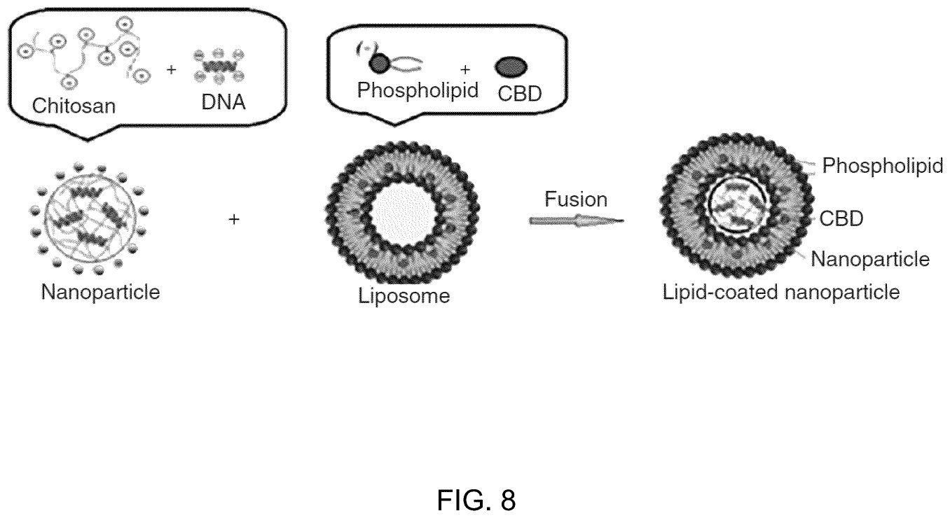

As used herein, “core” refers to a central portion of a nanoparticle, wherein the nanoparticle comprises at least two layers, e.g., a core comprising charged polymers, e.g., chitosan or chitosan lactate; a payload, e.g., an siRNA; and a phospholipid outer layer. See, e.g., FIG. 8 which shows an exemplary embodiment of the instant disclosure wherein the core comprises chitosan and an siRNA and the outer layer comprises a liposome comprising phospholipids and cannabidiol (CBD).

Suitable phospholipids for use in the disclosed compositions may include, but are not limited to, 1,2-di-(9Z-octadecenoyl)-sn-glycero-3-phosphocholine (DOPC), 1,2-dipalmitoyl-sn-glycero-3-phosphocholine (DPPC), 1,2-dipalmitoyl-sn-glycero-3-phosphoethanolamine-N-[3-(2-pyridyldithio) propionate] (PDP-PE), 3060i10, tetrakis (8-methylnonyl) 3,3′,3″,3″-(((methylazanediyl) bis (propane-3,1 diyl)) bis (azanetriyl)) tetrapropionate; 9A1P9, decyl (2-(dioctylammonio) ethyl) phosphate; A2-Iso5-2DC18, ethyl 5,5-di((Z)-heptadec-8-en-1-yl)-1-(3-(pyrrolidin-1-yl) propyl)-2,5-dihydro-1H-imidazole-2-carboxylate; ALC-0315, ((4-hydroxybutyl) azanediyl) bis (hexane-6,1-diyl) bis (2-hexyldecanoate); ALC-0159, 2-[(polyethylene glycol)-2000]-N,N-ditetradecylacetamide; β-sitosterol, (3S,8S,9S,10R,13R,14S, 17R)-17-((2R,5R)-5-ethyl-6-methylheptan-2-yl)-10,13-dimethyl-2,3,4,7,8,9,10,11,12,13,14,15,16,17-tetradecahydro-1 H-cyclopenta [a] phenanthren-3-ol; BAME-O16B, bis (2-(dodecyldisulfanyl) ethyl) 3,3′-((3-methyl-9-oxo-10-oxa-13,14-dithia-3,6-diazahexacosyl) azanediyl) dipropionate; BHEM-Cholesterol, 2-(((((3S,8S,9S,10R,13R,14S,17R)-10,13-dimethyl-17-((R)-6-methylheptan-2-yl)-2,3,4,7,8,9,10,11,12,13,14,15,16,17-tetradecahydro-1H-cyclopenta [a] phenanthren-3-yl) oxy) carbonyl) amino)-N,N-bis (2-hydroxyethyl)-N-methylethan-1-aminium bromide; C12-200, 1,1′-((2-(4-(2-((2-(bis (2-hydroxydodecyl) amino) ethyl) (2-hydroxydodecyl) amino) ethyl) piperazin-1-yl) ethyl) azanediyl) bis (dodecan-2-ol); cKK-E12, 3,6-bis (4-(bis (2-hydroxydodecyl) amino) butyl) piperazine-2,5-dione; DC-Cholesterol, 3β-[N-(N′,N′-dimethylaminoethane)-carbamoyl]cholesterol; DLin-MC3-DMA, (6Z,9Z,28Z,31Z)-heptatriaconta-6,9,28,31-tetraen-19-yl 4-(dimethylamino) butanoate; DOPE, 1,2-diolcoyl-sn-glycero-3-phosphoethanolamine; DOSPA, 2,3-dioleyloxy-N-[2-(sperminecarboxamido) ethyl]-N,N-dimethyl-1-propanaminium trifluoroacetate; DOTAP, 1,2-dioleoyl-3-trimethylammonium-propane; DOTMA, 1,2-di-O-octadecenyl-3-trimethylammonium-propane; DSPC, 1,2-distearoyl-sn-glycero-3-phosphocholine; cPC, ethylphosphatidylcholine; FTT5, hexa (octan-3-yl) 9,9′,9″,9″′,9″″,9′″″-((((benzene-1,3,5-tricarbonyl) yris (azanediyl)) tris (propane-3,1-diyl)) tris (azanetriyl)) hexanonanoate; Lipid H (SM-102), heptadecan-9-yl 8-((2-hydroxyethyl) (6-oxo-6-(undecyloxy) hexyl) amino) octanoate; OF-Deg-Lin, (((3,6-dioxopiperazine-2,5-diyl) bis (butane-4, 1-diyl)) bis (azanetriyl)) tetrakis (ethane-2,1-diyl) (9Z,9′Z,9″Z,9″Z,12Z,12′Z, 12″Z, 12″Z)-tetrakis (octadeca-9,12-dienoate); PEG2000-DMG, 1,2-dimyristoyl-rac-glycero-3-methoxypolyethylene glycol-2000; TT3, N¹,N³, N⁵-tris (3-(didodecylamino) propyl) benzene-1,3,5-tricarboxamide. Suitably, the phospholipids comprise DOPC, DPPC, or DOPC and DPPC.

The novel and non-obvious disclosed nanoparticles effectively deliver both siRNAs and anti-inflammatory compounds to cells. Exemplary anti-inflammatory compounds include, but are not limited to, cannabidiol (CBD), and delta-9-tetrahydrocannabinol (THC). THC may be used alone or in combination with CBD to be included in the lipid shell. It is envisioned that THC may provide neuroprotection as well as other properties useful for the treatment of the neurodegenerative diseases, e.g., anxiolytic or sedative properties. The anti-inflammatory compounds may be non-polar or have low bioavailability. The disclosed nanoparticle compositions advantageously have both charged and non-polar layers, as well as the ability to deliver compounds to cells directly, which may be leveraged to improve solubility/bioavailability of payload compounds.

It was determined that a final ratio of CS and/or CSL:DPPC:DOPC:CBD of about 20:12:10.5:7.5:1 leads to excellent knockdown of HTT as well as anti-inflammatory properties. In another embodiment, the ratio of chitosan lactate (CSL):DPPC:DOPC:CBD may be about 20:12:10.5:7.5:1.

### Pharmaceutical Compositions

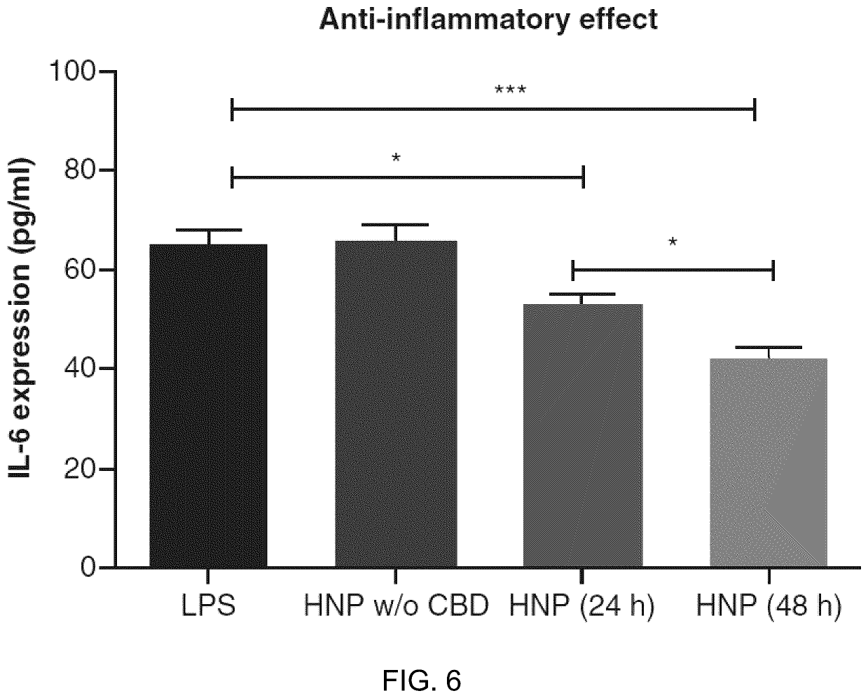

It is demonstrated that the disclosed compositions are effective in knocking down mRNA expression (FIG. 4) as well as delivering anti-inflammatory compounds directly to cells (FIG. 6).

Accordingly, in another aspect of the current disclosure, pharmaceutical compositions are provided. The pharmaceutical compositions comprise the disclosed siRNAs or nanoparticles and, optionally, a pharmaceutically acceptable carrier or excipient. Suitable methods of preparing pharmaceutical compositions, e.g., formulations, are known in the art.

Exemplary formulations of pharmaceutical compositions of the instant disclosure include, but are not limited to, formulations for intranasal, intravenous, intracranial, and intrathecal administration. As the disclosed siRNAs target HTT, formulations of the disclosed pharmaceutical compositions should suitably be made that allow for delivery to the brain. In this respect, it is envisioned that formulations for intranasal administration may be particularly advantageous, though the instant pharmaceutical compositions are not intended to be limited to intranasal formulations.

### Methods

In another aspect of the current disclosure, methods are provided. In some embodiments, the methods comprise administering a therapeutically effective amount of the disclosed pharmaceutical compositions to a subject in need thereof.

As used herein, a subject in need thereof may be a subject suffering from a neurological disease or disorder, e.g., a neurodegenerative disease or disorder. The neurodegenerative disease or disorder may be, but is not limited to, Huntington's disease, familial Parkinson's disease, familial Alzheimer's disease, or familial amyotrophic lateral sclerosis.

The present disclosure demonstrates that the disclosed nanoparticles can induce mRNA knockdown by delivery of an siRNA as well as reduce inflammation pharmacologically with anti-inflammatory compounds, e.g., CBD (FIGS. 4 and 6). Therefore, in another aspect of the current disclosure, methods of reducing expression of a gene related to a neurological disease or disorder and/or reducing inflammation in a subject in need thereof are provided. The neurodegenerative disease or disorder may be, but is not limited to, Huntington's disease, familial Parkinson's disease, familial Alzheimer's disease, or familial amyotrophic lateral sclerosis.

As used herein, “administration” may refer to administration of the disclosed compositions through any route that is pharmaceutically acceptable and is determined at the discretion of a physician. However, exemplary routes of administering the disclosed pharmaceutical compositions comprise intranasal administration and intravenous administration. It is envisioned that the disclosed nanoparticles, in particular, may be administered intranasally to treat neurological disease or disorders, e.g., Huntington's disease, as the disclosed nanoparticles may be directed, by administration intranasally, to cells expressing disease causing huntingtin mRNA, expression of which may be knocked down by the disclosed compositions. Further exemplary routes of administering the disclosed compositions comprise oral, transdermal, percutaneous, intravenous, intracranial, intramuscular, buccal, and/or intrathecal administration.

In another aspect of the current disclosure, methods of making the disclosed nanoparticles are provided. In some embodiments, the methods of making the nanoparticles comprise: making the polymer core by gelation of CS or CSL polymer with oligonucleotide under vigorous stirring at room temperature at a final amine-to-phosphate (N: P) ratio of 2:1 (CS/oligonucleotide) and 1:1 (CSL/oligonucleotide; making the liposomes by mixing DPPC and DOPC and CBD stock solution (5:2.5:1 mol %) with 2:1 (v/v) chloroform and methanol; evaporating the organic solvent, i.e., chloroform, hydrating the composition, followed, optionally, by mixing and extruding the liposomes to reduce their size; fusing the liposomes and polymer cores by adding an appropriate amount of liposome solution to NP solution at a mass (w/w) ratio of 1:2 and mixing the solution.

### Definitions

The present disclosure is further described herein using several definitions, as set forth below and throughout the application.

The disclosed subject matter may be further described using definitions and terminology as follows. The definitions and terminology used herein are for the purpose of describing particular embodiments only and are not intended to be limiting.

The phrases “% sequence identity,” “percent identity,” or “% identity” refer to the percentage of amino acid residue matches between at least two amino acid sequences aligned using a standardized algorithm. Methods of amino acid sequence alignment are well-known in the art. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B, and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or B″ or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### EXAMPLES

The following Examples are illustrative and should not be interpreted to limit the scope of the claimed subject matter.

### Example 1-Dual-Function Hybrid Nanoparticles with Gene Silencing and Anti-Inflammatory Effects

### Methods

### Materials

Stock ampules (25 mg) of dipalmitoylphosphatidylcholine (DPPC) (purity >99%) and 1,2-dioleoyl-sn-glycero-3-phosphocholine (DOPC) (purity >99%) chloroform solutions were purchased from Avanti Polar Lipids (AL, USA) and stored at −20° C. The stock ampules of CBD (1 mg), chloroform (purity >99.8%), chloroform and methanol (purity >99.8%), low-molecular-weight CS (50,000-190,000 Da with 85% deacetylation), CS lactate (CSL; 5000 Da), lipofectamine 3000, Nile red, calcein and Hoechst 33342 fluorophores were purchased from Sigma-Aldrich (MO, USA). Mangafodipir (MFDP) was purchased from the US Pharmacopeial Convention (MD, USA). Avanti Mini Extruder and polycarbonate membranes were purchased from Avanti Polar Lipids.

OptiMEM I reduced serum medium, RPMI (Roswell Park Memorial Institute) 1640, GlutaMAX, minimum essential medium nonessential amino acid solution, amphotericin B, Luria-Bertani broth, kanamycin, ampicillin, HEPES buffer solution, sodium pyruvate, penicillin-streptomycin, fetal bovine serum, phosphate-buffered saline (pH 7.4), trypan blue (0.4%) and trypsin-EDTA were obtained from Invitrogen (CA, USA). Tissue culture flasks (75 and 25 cm2) and 96-well plates were purchased from Alkali Scientific (FL, USA), and six- and 24-well CytoOne tissue culture plates were purchased from USA Scientific (FL, USA).

Zyppy Plasmid Miniprep Kit and ZymoPURE Plasmid Maxiprep Kit were purchased from Sigma-Aldrich. TaqMan™ Fast Advanced Master Mix and primers for quantitative PCR were obtained from Thermo Fisher Scientific (MA, USA), including HTT (assay identifier [ID]: Hs00918174 ml and Mm01213820 ml) and PPIB (assay ID: Mm00478295 ml) as a reference gene. Mouse IL-6 ELISA kit, lipopolysaccharide (LPS) from Escherichia coli serotype 0111: B4 and dexamethasone were purchased from Sigma-Aldrich. All other chemicals and reagents used were also of analytical grade. UltraPure™ DNase/RNase-Free Distilled Water, purchased from Thermo Fisher Scientific, was used for all experiments.

### Oligonucleotides

### Plasmid DNA

E. coli transformed with plasmid DNA (mCherry plasmid) were inoculated into Luria-Bertani broth medium at a ratio of 1 μl for 1 ml of medium (kanamycin 50 mg/ml, ampicillin 100 mg/ml) for 20-24 h at 37° C. Plasmid DNA was isolated using a Zyppy Plasmid Miniprep Kit. A ZymoPURE Plasmid Maxiprep Kit was used to purify transfection-grade plasmid DNA. Plasmid DNA concentration was determined by UV-Vis measurements using a NanoDrop™ 2000 Spectrophotometer (Thermo Fisher Scientific). The purity of plasmid DNA was >95%.

siRNA

Three different siRNA compounds were packaged into NPs or liposomes. Silencer Select siRNA s6491 was purchased from Thermo Fisher Scientific. Scrambled siRNA, which has no homology to any known mammalian genes, was used as the no template control (NTC) in the knockdown experiments. Novel siRNA targeting exon 30 of HTT mRNA (E30) was designed using the RefSeq sequence NM 002111.8. This siRNA was designed in the inventors' laboratory with the use of theoretical basics, web-based tools, and open-access databases. E30 was labeled with Cy3 and was used to assess the efficiency of siRNA uptake. The designed siRNA and NTC were synthesized, modified (siSTABLE) and purified by Dharmacon Inc. (CO, USA), a Horizon Discovery Group company.

### Nanoparticles

### Polymeric NP Preparation

Polymeric NPs were fabricated via ionotropic gelation of CS polymer with oligonucleotide under vigorous stirring at room temperature. The fabrication technique used by the inventors has been described previously [2]. The optimal NPs were formulated to obtain a final amine-to-phosphate (N: P) ratio of 2:1 (CS/oligonucleotide) and 1:1 (CSL/oligonucleotide).

### Liposome Preparation

The liposomes were prepared according to the classical film hydration method. Phospholipids DPPC and DOPC and CBD stock solution (5:2.5:1 mol %) with 2:1 (v/v) chloroform and methanol in 2 ml were mixed in an amber glass bottle. Nile red dissolved in chloroform was used at stock concentration (100 μg/ml). A thin lipid film was obtained by evaporation of organic solvent under the vacuum and hydrated in distilled, deionized water. To obtain liposomal suspension, the components were mixed by magnetic stirrer for 30 min at 1100 r.p.m. at moderate heat. Heat and time are reciprocal factors for the generation of the disclosed liposomes. As used herein, “moderate heat,” is used to emphasize that excessive heat can cause undesirable oxidation. This was followed by sonication or extrusion (11 times) through 200-nm polycarbonate membranes (Whatman Filters purchased from MilliporeSigma, MO, USA) using an Avanti Mini Extruder to reduce their size. The final liposomal composition was DPPC 58%, DOPC 30% and CBD 12%.

### HNP Preparation

HNPs were prepared by fusion of liposomes and NPs by adding an appropriate amount of liposome solution to NP solution at a mass (w/w) ratio of 1:2. The mixture was stirred for 1 h at 1100 r.p.m. above phase transition temperature. The final HNP composition was CS/CSL 40%, DPPC 24%, nucleic acid 21%, DOPC 13% and CBD 2%.

### NP Characterization

The size and (potential of NPs, liposomes and lipid-coated NPs were measured using a Zetasizer Nano ZS (Malvern Panalytical Ltd., Malvern, UK). Based on liposomal size, large unilamellar vesicles (>100 nm) were obtained [19]. Interaction, cation/nucleic acid molar and NP ratios of full condensation of DNA by each nanocarrier were confirmed by gel retardation assay.

Loading efficiency was measured based on quantitative fluorescence detection using hydrophilic and hydrophobic dyes. To stain plasmid DNA NPs, Hoechst 33342 blue fluorescent dye was used (excitation: 350 nm; emission: 462 nm), and Nile red, a lipophilic fluorophore, was used for lipid staining (excitation: 552; emission: 639 nm). Hoechst 33342 intercalating agent stock solution was added to plasmid DNA solution before NP preparation. Fluorescence readings of samples were performed in 96-well plates using a Synergy H1 Hybrid Multi-Mode Reader (BioTek Instruments, VT, USA). Loading efficiency was quantified based on the change in fluorescence as a result of liposomal shell formation around free or NP-encapsulated DNA using the equation LE %=100−((F1/F2)×100), where F1 is the fluorescence of the DNA and F2 is the fluorescence of the appropriate lipid shell. The results are expressed as mean ±standard deviation (SD) (n=3).

### Cell Culture

Bone marrow mesenchymal stem (BMMS) cells were generated from 4- to 6-month-old transgenic YAC128 mice (also known as the FVB-Tg YAC128/53Hay/J line) using previously described methods^{[20.21]}. All procedures with animals were performed in accordance with the Institutional Animal Care and Use Committee-approved protocol. YAC128 transgenic and YAC128 noncarrier mice (stock no. 004938) were purchased from The Jackson Laboratory (ME, USA), and breeding colonies were established in a University of South Florida comparative medicine animal facility. The mice were housed under standard conditions with free access to water and food.

Mice were killed by cervical dislocation and sterilized in 70% ethanol and then placed on clean aluminum foil inside the biosafety cabinet. Hind limbs were detached from the body by cutting the caudal bones along with the femora. Skin and muscles along the bones were removed from the limbs. Feet and tibiae underwent full removal. Both ends of the femoral bones were cut off with scissors so that the marrow cells could be flushed out. The tip of a 26-gauge needle (attached to a 5-ml syringe filled with complete RPMI 1640 medium) was placed into the proximal end of each femur to flush the marrow through the bone. The flushed material was collected in a 50-ml sterile Falcon tube (Corning, NY, USA) and passed through a 22-gauge needle three to four times to make a single-cell suspension. Tubes with cells underwent centrifugation (700 g for 5 min at room temperature) and medium replacement, and cells were then seeded in a 25-cm2 flask at a concentration of 2 million cells/ml. On the third and fifth days, two-thirds of the medium was replaced with fresh growth medium. On the seventh day, the cells reached 90% confluency, and treatment with 1 ml of 0.25% trypsin was performed for 5 min at 37 C [21]. The BMMS cells were then used for functional experiments. The cells were cultured in complete RPMI 1640 medium supplemented with 10% heat-inactivated fetal bovine serum, 10 mM HEPES, 1 mM sodium pyruvate, 1× penicillin-streptomycin, 0.25 μg/ml amphotericin B, 1× nonessential amino acids, and 1× GlutaMAX and maintained at 37 C and 5% CO₂ under humid conditions.

### Assessment of Cell Viability

Bone marrow mesenchymal stem (BMMS) cells were dispensed in 24-well plates at a density of 1×10⁵ cells per well and treated with plasmid DNA loaded into NPs, HNPs, and Lipofectamine 3000-based liposomes (dose: 0, 2, 4, 8 and 10 μg/ml) for 24 h. Cells were detached by trypsinization, and the number of viable cells was counted using a 0.4% trypan blue staining reagent. The viability of the control (untreated cells) was regarded as 100%.

### Transfection & Cellular Uptake Evaluation

BMMS cells were seeded onto 24-well plates at a density of 1×10⁵ cells per well and incubated for 48 h before transfection. NPs, HNPs, and Lipofectamine 3000-based liposomes containing plasmid DNA were added in triplicate to the wells with OptiMEM medium, and the plates were gently swirled (dose: 4 μg/ml). As a positive control, the commercial transfection reagent Lipofectamine 3000 was used. Fresh growth medium was added to the wells after incubation for 6 h at 37 C and 5% CO₂. The cells were left in the incubator for 24 h before being further assayed and monitored for 5 days. Each experiment was performed in triplicate, and the results are presented as the mean of these experiments. The number of positive cells within a transfected cell population was monitored and determined using a VHX-700 digital fluorescence microscope (Keyence Corporation, Osaka, Japan). Transfection efficacy was measured using ImageJ image analysis software.

The ability of NPs and HNPs to deliver Cy3-labeled siRNA E30 into BMMS cells was investigated. A six-well plate was prepared with the BMMS cell line (2.5×10⁵ cells per well) and cultured at 37 C for 48 h in regular medium. The culture medium was changed to OptiMEM for transfection performance. E30-loaded nanoformulations were diluted in OptiMEM and added to the cells (dose: 2.5 μg/ml). After incubation for 6 h, cells were washed twice with phosphate-buffered saline and incubated in regular medium. To stain cells and nuclei, the working solution of indicators in complete medium (1 μM calcein and 1 μM Hoechst 33342) was added to each well and incubated for 30 min at room temperature. Cells were studied without fixation by fluorescence microscopy. The imaging conditions were kept constant for observation of the different samples, and images were taken with a VHX-700 digital microscope.

### Assessment of CBD Anti-Inflammatory Effect

BMMS cells were seeded onto 24-well plates at a density of 1×10⁵ cells per well and incubated for 48 h. Cells were stimulated by replacing culture medium with a similar volume of OptiMEM medium containing 1 μg/ml LPS or LPS plus 1 μg/ml dexamethasone or LPS in combination with liposomes loaded with CBD (1 μg/ml). All conditions were tested in triplicate. The conditioned medium was collected after 48 h of incubation at 37 C and kept frozen until assayed for cytokines by mouse IL-6 ELISA.

### Assessment of HTT Gene Silencing

To investigate the ability of each siRNA to downregulate expression of the target gene, BMMS cells were seeded onto 25-cm2 cell culture flasks at a density of 4×10⁶ cells per flask and incubated for 48 h before transfection. CSL-based NPs, HNPs and Lipofectamine 3000-based liposomes loaded with siRNAs were diluted with OptiMEM medium and added to the flasks. The amount of siRNA was 10 μg per flask (2.5 μg/ml). The cells were kept in an incubator for 24 h at 37° C. and 5% CO₂.

Total RNA from BMMS cells was extracted with the use of TRIzol reagent and Phasemaker™ tubes (Thermo Fisher Scientific). RNA was reverse transcribed using an Invitrogen™ SuperScript™ III Reverse Transcriptase Kit with Invitrogen™ Oligo (dT) 20 Primer (Thermo Fisher Scientific). Levels of mRNA expression were measured using QuantStudio 3 (Thermo Fisher Scientific). TaqMan™ Fast Advanced Master Mix was used for real-time quantitative PCR, which was carried out in accordance with the manufacturer's protocol. Expression levels of HTT mRNA were normalized to PPIB mRNA levels. Primers for quantitative PCR were obtained from Thermo Fisher Scientific, including HTT (assay ID: Hs00918174 ml and Mm01213820 ml) and PPIB (assay ID: Mm00478295 ml).

### Statistical Analysis

Mean, SD, standard error of the mean and p-values were calculated using Prism 5 (GraphPad Software Inc., CA, USA). Results are expressed as mean+SD or standard error of the mean. BMMS cell culture HTT mRNA expression is expressed as mean RQ-1+SD (n=3-4). Statistical analysis utilized two-way analysis of variance or one-way analysis of variance with correction for multiple comparisons using Prism 5.

### Results

### Physicochemical Characterization of NPs

NPs and HNPs, produced as described earlier, were analyzed by dynamic light scattering and demonstrated a relatively narrow particle size distribution (range: 150-210 nm). The liposomes used in this study were sized down and homogenized using sonication or extrusion, which made these liposomal vesicles suitable for encapsulation of NPs inside the hydrophilic core [22]. The I potentials of CS- and CSL-based NPs were similar (41.1+3.5 and 50.1+5.3, respectively). HNPs were characterized by a reduced surface charge (7.97+3.4) as a result of the anionic nature of the lipids used for formation of the outer lipid shell around the NPs.

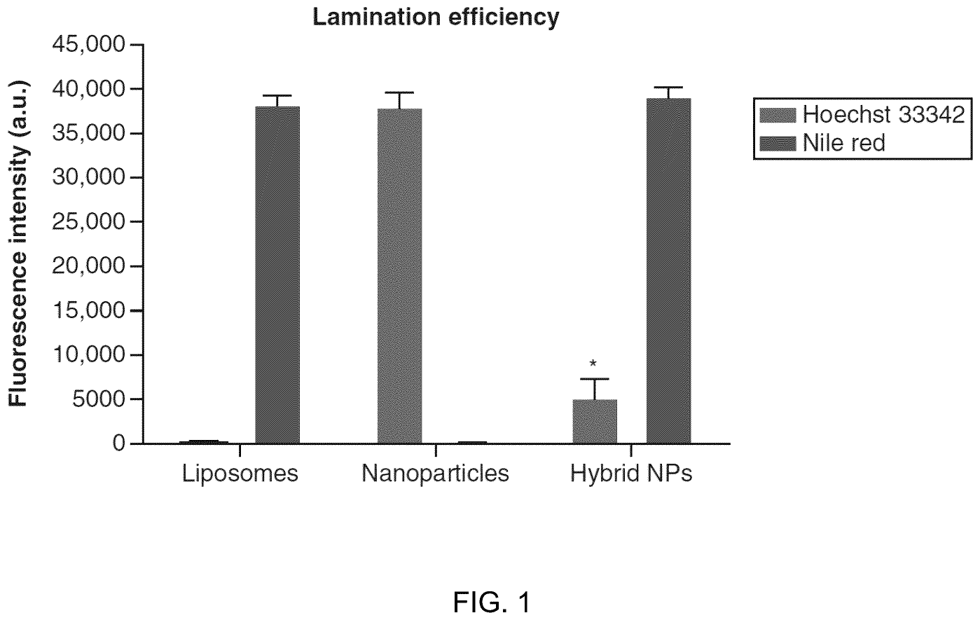

To quantitatively confirm the formation of the lipid shell on the polymer/plasmid DNA NP surface, the entrapment efficacy was determined using fluorescence intensity data. The liposomes were marked with Nile red fluorescent dye, and NPs loaded with plasmid DNA were labeled with Hoechst 33342 intercalating agent (FIG. 1). The integrity of the hybrid complex, as measured by gel retardation assay after the fusion of liposomes and NPs, was confirmed by the absence of plasmid DNA release from the HNPs. A significant reduction in Hoechst 33342 fluorescence intensity was observed in NPs compared with HNPs (FIG. 1). Nile red fluorescence was not reduced in either liposomes or HNPs. This can be considered to represent lamination of polymeric NPs with a lipid layer. The loading efficiency of NPs by liposomes was 85.8+10.8%. The characteristics of the nanostructures used in this study are represented in Table 1.

### Effect of NP Lamination on Cytotoxicity

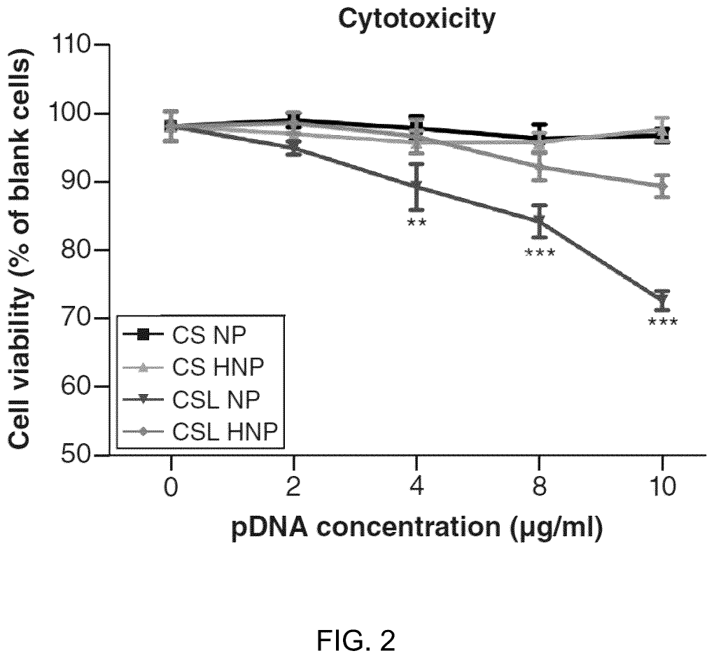

In this study, the inventors used a transgenic BMMS cell line derived from transgenic FVB-Tg YAC128/53Hay/J mice to generate a cell model that expresses mutant human HTT mRNA. The BMMS cell line was used in preliminary testing for selection of the nanocarrier as well as HTT silencing and to assess anti-inflammatory effects. The use of CS and CSL NPs for siRNA delivery in lowering HTT expression following intranasal administration has been previously described [3]. Here the inventors evaluated the toxicity of our novel hybrid nanocarrier systems on BMMS cells in comparison with the toxicity of polymeric NPs (FIG. 2). The cytotoxicity of all preparations was studied 24 h post-treatment over a range of loaded plasmid DNA concentrations.

CSL-based NP incubation significantly decreased BMMS viability, but incubation with CSL-based HNPs had minimal impact on cellular viability. At lower plasmid DNA concentrations, the difference between the toxicities of CSL NPs and CSL HNPs was minimal, but upon increasing the plasmid concentration, HNPs showed a much better toxicity profile (FIG. 2). This demonstrated that lamination of the NPs with liposomes had a substantially positive effect on reducing cytotoxicity. In further experiments, mCherry plasmid concentration was limited to 4 μg/ml.

### Comparative Transfection Efficacy of NPs

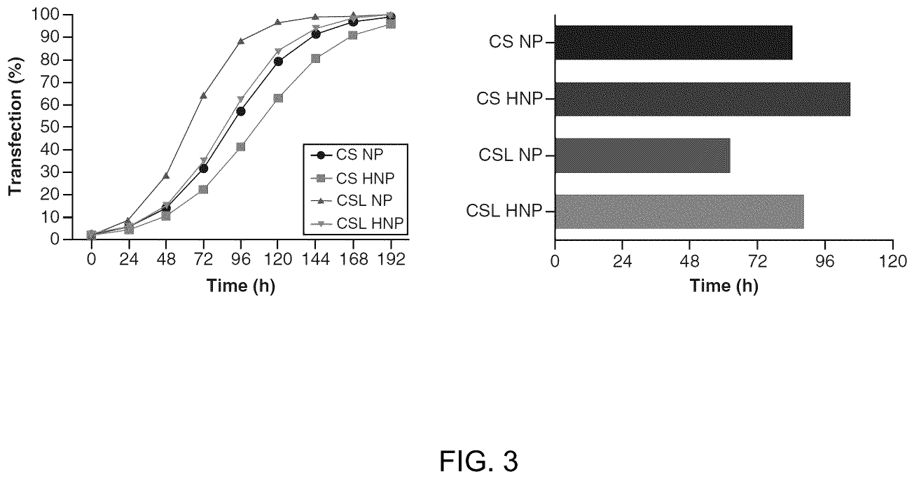

The transfection efficacy of NPs and HNPs was evaluated by mCherry red protein expression. Fluorescence of the red protein was measured in BMMS cells for the tested nanostructures over 96 h, and transfection efficacy was normalized in relation to Lipofectamine 3000, which was employed as a standard transfection reagent, and expressed as a percentage. The results of comparative transfection are presented in Table 2. Tested nanostructures demonstrated different rates of transfection that could be calculated from kinetic curves represented by the logistic model:

$$F \% = \frac{100}{( 1 + 50 {e}^{( \frac{R T}{24} )} )}$$

where F % is the percentage of mCherry expression, parameter R represents the individual fitting coefficient selected for each kinetic dataset (Table 2) and T is the time point of the observation. FIG. 3 shows the kinetics of the calculated expression of mCherry carried by different nanostructures.

Extrapolation of experimental data with the proposed logistic formula allowed determination of the 50% transfection level for comparative analysis of tested nanostructures (FIG. 4). At the 50% transfection level, CS NPs and CSL HNPs demonstrated similar transfection rates (between 85 and 89 h). CSL NPs showed the highest performance, reaching a 50% level of transfection at 62.6 h, and CS HNPs demonstrated the lowest performance, requiring 105.5 h to approach the same level.

HNPs exhibited a reduced transfection efficacy in comparison with polymeric NPs, although CSL HNPs were found to be a more effective gene-delivery agent than CS NPs (FIG. 4). Incubation of HNPs resulted in delayed expression of mCherry red fluorescent protein. These HNPs exhibited a prolonged sustained release profile due to the slow degradation and deprotonation rate of the polymer in the core and the diffusion barrier of the lipid shell. HNPs provide the advantages of both polymeric NPs and liposomes, making it possible to incorporate drugs with different physiological characteristics, resulting in greater stability without drug leaking and optimal benefits with minimal toxicity. It is important to note that the highest transfection efficacy is preferable for cell culture experiments. However, for in vivo transfection, this is not always suitable because payload will be depleted before the drug can reach the target and therefore may contribute to the development of undesirable off-target effects.

### HTT Gene Silencing Effects

Here the inventors used two different anti-HTT siRNAs: commercially available s6491 obtained from Thermo Fisher Scientific and a novel oligonucleotide produced in the inventors' laboratory (E30 siRNA). The E30 siRNA was designed in the inventors' laboratory using recommendations published elsewhere^{[10,23-28]}. E30 siRNA was selected from multiple candidates with the least ‘off-target’ effects. The Basic Local Alignment Search Tool (BLAST) was used to search the National Center for Biotechnology database and Ensembl BLAST/BLAST-like alignment tool search database were used to determine homology across gene families and species and filter all siRNA candidates to remove nonunique sequences. The off-target score was evaluated for each cross-reactive siRNA based on the degree of similarity to transcripts in the databases^{[29]}. The designed siRNA sequence E30 (Table 3) was synthesized by Dharmacon Inc., and the dual-strand modification pattern siSTABLE was implemented, including SS modification to prevent interaction with RISC and favor antisense strand uptake and AS seed region modification to destabilize off-target activity and enhance target specificity. An SS double overhang was synthesized with UU at the 3′ end.

The efficacy of HTT gene silencing for siRNA E30 was tested in comparison with s6491 commercial siRNA, which was employed as a reference. The scrambled siRNA was used as NTC. FIG. 3 illustrates the downregulation of HTT expression in BMMS cells 24 h after Lipofectamine 3000-mediated delivery of two siRNAs at a dose of 2.5 μg/ml. The siRNA s6491 significantly suppressed expression of the mutant HTT mRNA. The reduction in HTT expression reported here demonstrated that E30 siRNA has the ability to lower mutant HTT gene expression with efficacy similar to that observed for s6491 siRNA.

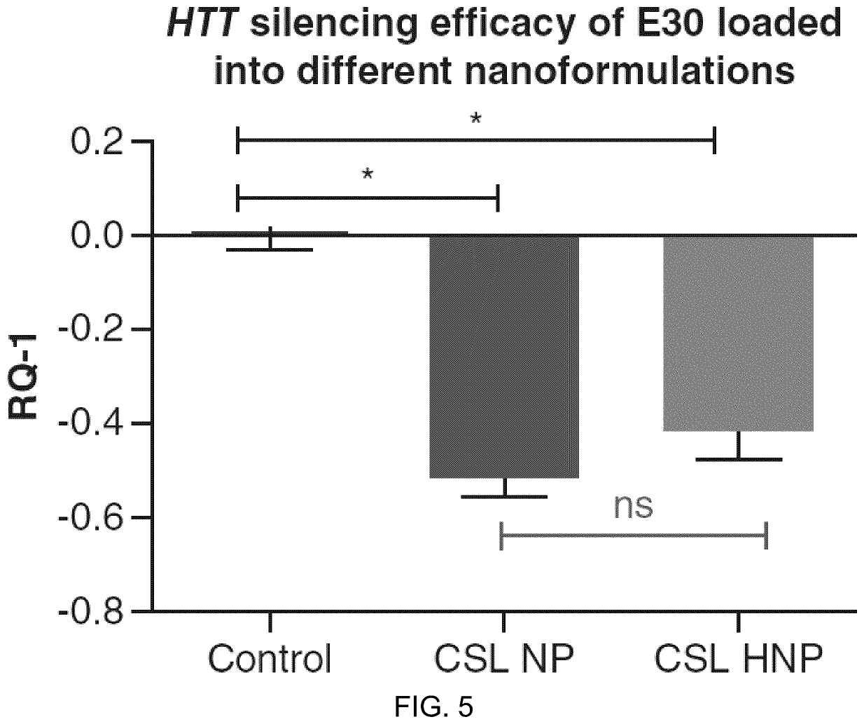

Assessment of mRNA knockdown level mediated by the inventors' NPs and HNPs is illustrated in FIG. 5. There was no significant difference in HTT mRNA silencing levels with E30 loaded into CSL NPs and CSL HNPs. As expected, NPs containing NTC did not produce HTT downregulation.

HNPs with Lipid Shell Containing CBD Effectively Suppressed Inflammation

To assess the anti-inflammatory potential of CBD-loaded, CSL-based HNPs, the BMMS cell line was used (FIG. 6). A moderate degree of inflammation was induced in the cells by incubating the cultures with LPS, as indicated by expression of the inflammatory cytokine IL-6. The anti-inflammatory activity values of CBD-carrying HNPs were assessed by measuring IL-6 levels in the cell cultures 24 and 48 h after treatment. The corticosteroid dexamethasone (1 μg/ml) was used as a positive control in this study.

### Cellular Internalization of HNPs

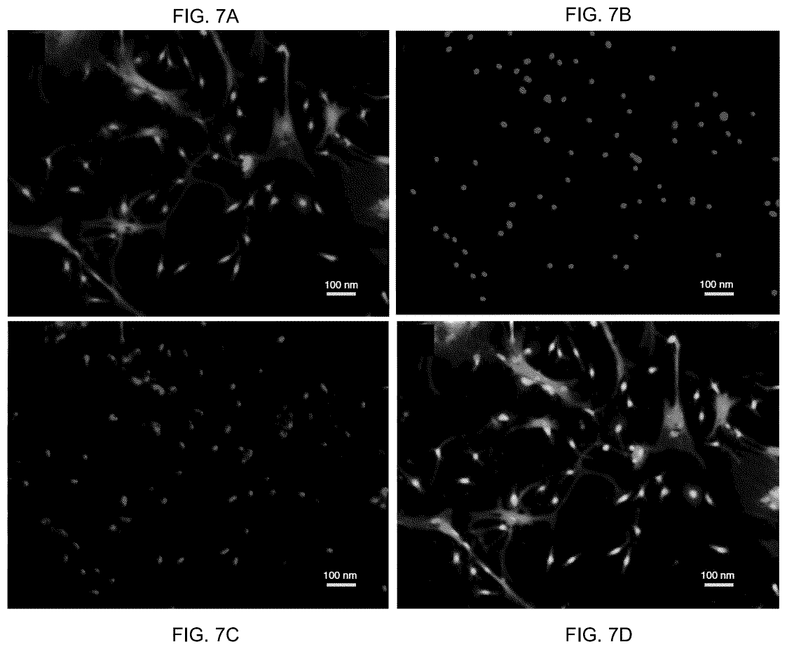

The success of siRNAs as therapeutics for brain diseases is largely dependent on the development of a delivery vehicle that can safely and efficiently deliver the therapeutic agent in vivo from nose-to-brain. In addition, the delivery vehicle should facilitate cellular internalization of the payload. To investigate the extent of cellular internalization of HNPs, BMMS cell cultures were used to monitor the cellular uptake of CSL-based HNPs loaded with Cy3-labeled siRNA, as assessed by digital fluorescence microscopy. The results presented in FIG. 7 reveal that 6 h after HNP delivery of Cy3-labeled siRNA, fluorescent particles were found to be located inside each BMMS cell.

### DISCUSSION

Many therapeutic agents, especially nucleic acids like siRNA, require protection against rapid chemical or enzymatic degradation and need to be efficiently transported across the blood-brain barrier to targets in the central nervous system (CNS). Therefore, as a result of the rapid degradation of nucleic acids, poor penetrability across the blood-brain barrier and limited cellular uptake, the potential of siRNA-based therapeutics in gene therapy for HD and other brain diseases remains a challenging problem^{[30]}. Although specific chemical modifications of siRNAs provide resistance to nuclease degradation, the negatively charged siRNA molecules are unable to cross the cell membrane and other biological barriers, which limits their clinical applications^{[2,30-32]}. Development of an effective delivery system for siRNA-mediated gene therapy has great potential to alter the course of diseases like HD. Positively charged nanocarriers facilitate spontaneous electrostatic interactions with nucleic acids as well as binding of the resulting nanoformulations to the negatively charged components of the cell membrane prior to cellular internalization of therapeutic genes or gene silencing molecules. In brief, NPs and liposomes are well suited to overcoming the obstacles that limit drug and gene delivery^{[33,34]}. Liposomes have gained attention mainly because of their ability to improve the delivery of genes to target cells. However, the chemical stability of liposomes is often contested, and NPs, because of their polymeric matrix, are more stable than liposomes in biological fluids, have a longer shelf life and provide safe and effective delivery of packaged payloads to the brain^{[30,35]}.

As described earlier, a new HTT gene silencing agent, siRNA E30, was produced, packaged in various nanocarriers and tested in the BMMS cell culture line, which carries the human HTT gene mutation. Previously, the inventors reported on the use of CS- and CSL-based NPs for intranasal delivery of gene therapy for HD^{[2]}. These two nanocarriers were assessed for transfection efficacy and cytotoxicity. All NP formulations exhibited time-dependent transfection capacity. There was an essential difference in transfection efficacy between the tested NPs, and this was more evident in comparison with Lipofectamine 3000. The inventors found that the highest transfection was achieved with CSL NPs (FIG. 4), whereas cells treated with CS NPs expressed the fluorescent protein only at 48 h post-treatment. Cell survival was not affected by the amount of nanocarriers used for transfection (NPs loaded with 4 μg of plasmid DNA), with viability values around 95%. Cell viability studies did not show any observable toxicity effects resulting from CS NPs. However, CSL NPs demonstrated dose-dependent toxicity (FIG. 2).

To improve the safety profile of CSL-based NPs, the lipid lamination approach was implemented to shield the NPs with a lipid shell^{[22]}. The liposome phospholipid bimolecular membrane is similar to the mammalian cell membrane, which promotes good biocompatibility and reduces immune response and toxicity (36). To construct liposomes, a combination of saturated (DPPC) and unsaturated (DOPC) phospholipids was used. The resulting liposomes had a low transition temperature (38° C.) and more free space within the bilayer, which allowed additional flexibility and, consequently, made them more permeable to water and other molecules. To stabilize the lipid membrane to water hydrolysis, lipophilic compounds were used, as they can intercalate into the phospholipid bilayer interface and displace water from the region^{[37-39]}. For this reason, the inventors used CBD to provide greater liposome stability as well as for its reported beneficial neuroprotective and anti-inflammatory activity^{[6,40,41]}. In the present study, the inventors demonstrated that these HNPs were effective in decreasing inflammation, as indicated by decreasing expression of the inflammatory cytokine IL-6 in cell cultures.

Application of polymeric nanocarriers in animal studies tends to be more effective than lipid-based delivery systems. However, the hybrid nanocarriers that exhibit complementary characteristics of both polymeric NPs and liposomes, particularly in terms of physiological stability and biocompatibility, have a better impact and much fewer limitations on in vivo drug delivery^{[42]}. The in vitro study of HTT downregulation in the cell cultures (FIG. 6) revealed slightly less downregulation of mutant HTT mRNAs following E30 delivery using CSL HNPs in comparison with CSL NPs. The data correlated with the transfection study (FIG. 4), showing improved cargo protection by the liposome molecular bilayer, as it shielded the NPs, thereby reducing degradation and prolonging the therapeutic effect. In addition, a significant advantage of the inventors' HNPs is the CBD therapeutic encapsulation within the lipid layer. The therapeutic value of CBD is its capacity to provide neuroprotection against a spectrum of neurotoxic mechanisms that operate in neurodegenerative disorders (excitotoxicity, inflammatory events, oxidative injury)^{[43]}.

Despite the obvious advantages of intranasal drug delivery, the nasal cavity presents a number of limitations for drug absorption, including low intrinsic permeability for some drugs, such as hydrophilic molecules^{[44]}. Thus, the lipophilic nature of HNPs promotes the permeation and delivery of siRNAs through the nasal mucosa. Chronic intranasal drug administration may result in nasal irritation and inflammation, but the CBD in the lipid shell of the HNPs can decrease tissue injury by modulating the cytokine biology of various cell systems and

decreasing inflammation^{[45]}. The in vitro studies reported here have shown the potent anti-inflammatory effect of HNPs loaded with cannabinoids. The use of dual-action HNPs to decrease expression of mutant HTT and decrease inflammatory changes is more likely to be effective in slowing disease progression in HD patients than administration of gene silencing agents alone.

### CONCLUSION

A dual-function HNP delivery system composed of a CS core loaded with novel anti-HTT siRNA and a lipid shell containing CBD has been designed and tested in the present disclosure. Physicochemical characterization of NPs demonstrated narrow particle size distribution (range: 150-210 nm) with reduced surface charge due to polymeric NP lamination with lipid composition. Lamination of polymeric NPs with a lipid shell containing CBD provided significant reduction in cytotoxicity. The transfection efficacy of NPs was dependent on the polymeric core structure and outer lipid shell, allowing variation in transfection time that should be beneficial for in vivo drug delivery. The novel dual-action hybrid nanocarrier system presently disclosed proved to be effective in both lowering gene expression and reducing inflammation in a BMMS cell line obtained from transgenic HD mice.

TABLE 1
Physical characteristics of fabricated lipid and polymeric
nanostructures and loading efficiency obtained after
fusion of liposomes with polymeric nanoparticles.

| | | Average t | |
|---|---|---|---|
| Nanostructure | Average size (nm) | potential (mV) | LE % |
| Liposome | 152.63 ± 15.69 | −3.73 ± 3 | |
| CS NP | 152.42 ± 18.67 | 41.1 ± 3.5 | |
| CSL NP | 175.67 ± 8.66 | 50.1 ± 5.3 | |
| CS HNP | 206.17 ± 16.27 | 7.97 ± 3.4 | 85.8 ± 10.8 |
| CSL HNP | 198.57 ± 12.72 | 6.29 ± 1.82 | 87.16 ± 9.69 |

Data are expressed as mean ± standard deviation (n = 3).
CS: Chitosan;
CSL: Chitosan lactate;
HNP: Hybrid nanoparticle;
LE %: Loading efficiency;
NP: Nanoparticle

TABLE 2
Expression of mCherry plasmid in bone marrow
mesenchymal stem cell line following transfection
with polymeric and hybrid nanoparticles.

| Time (h) | CS NP | CS HNP | CSL NP | CSL HNP |
|---|---|---|---|---|
| 24 | 6.6 ± 0.98 | 5 ± 0.61 | 14.7 ± 0.45 | 6.9 ± 0.89 |
| 48 | 18.69 ± 2.38 | 8.73 ± 1.05 | 29.63 ± 3.28 | 23.82 ± 4.59 |
| 72 | 31.17 ± 4.99 | 20.51 ± 3.51 | 65.33 ± 3.85 | 36.56 ± 1.68 |
| 96 | 56.43 ± 4.9 | 47.48 ± 1.48 | 90.41 ± 9.09 | 65.48 ± 2.75 |

The pDNA at a concentration of 4 μg/ml was equally used in each preparation, and results are given as transfection efficacy (protein expression of sample normalized to expression of cells transfected with Lipofectamine 3000).
Data are expressed as mean + standard error of the mean (n = 4).
CS: Chitosan; CSL: Chitosan lactate; HNP: Hybrid nanoparticle; NP: Nanoparticle; pDNA: Plasmid DNA.

TABLE 3
Characteristics of E30 siRNA in comparison
with s6491 commercial siRNA.

| siRNA name | siRNA structure | Target exon |
|---|---|---|
| S6491 | 5′-GAAUCGAGAUCGGAUGUCAtt-3′ | 11 |
| | \|\|\|\|\|\|\|\|\|\|\|\|\|\|\|\|\|\|\| | |
| | 3′-gaCUUAGCUCUAGCCUACAGU-5′ | |
| E30 | 5′-CUGCUUUAGUCGAGAACCAuu-3′ | 30 |
| | \|\|\|\|\|\|\|\|\|\|\|\|\|\|\|\|\|\|\| | |
| | 3′-agGACGAAAUCAGCUCUUGGU-5′ | |

| | |
|---|---|
| Cross-reactivity | Target location |

| siRNA | Homo | Mus | Homo | Mus |
|---|---|---|---|---|
| name | sapiens | musculus | sapiens | musculus |
| S6491 | 1 | 0 | 1515-1535 | |
| E30 | 1 | 1 | 4046-4066 | 4005-4025 |

Lower case letter indicates overhang nucleotides.

### REFERENCES

1. Babu A, Muralidharan R, Amreddy N, Mehta M, Munshi A, Ramesh R. Nanoparticles for siRNA-based gene silencing in tumor therapy. IEEE Trans. Nanobioscience 15 (8), 849-863 (2016).
2. Fihurka O, Sanchez-Ramos J, Sava V. Optimizing nanoparticle design for gene therapy: protection of oligonucleotides from degradation without impeding release of cargo. Nanomed. Nanosci. Res. 2 (6), 155 (2018).
3. Sava V, Fihurka O, Khvorova A, Sanchez-Ramos J. Enriched chitosan nanoparticles loaded with siRNA are effective in lowering Huntington's disease gene expression following intranasal administration. Nanomedicine 24, 102119 (2020).
4. Maroon J, Bost J. Review of the neurological benefits of phytocannabinoids. Surg. Neurol. Int. 9, 91 (2018).
5. Prenderville JA, Kelly AM, Downer EJ. The role of cannabinoids in adult neurogenesis. Br. J. Pharmacol. 172 (16), 3950-3963 (2015).
6. Atalay S, Jarocka-Karpowicz I, Skrzydlewska E. Antioxidative and anti-inflammatory properties of cannabidiol. Antioxidants (Basel) 9 (1), 21 (2019). Discusses the antioxidant and anti-inflammatory properties of cannabidiol (CBD) which is relevant in the present field of study.
7. Burstein S. Cannabidiol (CBD) and its analogs: a review of their effects on inflammation. Bioorg. Med. Chem. 23 (7), 1377-1385 (2015).
8. ·· Discusses the inflammatory effects of CBD with a focus on receptor binding, signaling events and downstream and functional effects.
9. Vuolo F, Petronilho F, Sonai B et al. Evaluation of serum cytokines levels and the role of cannabidiol treatment in animal model of asthma. Mediators Inflamm. 2015, 538670 (2015).
10. Ong SG, Ming LC, Lee KS, Yuen KH. Influence of the encapsulation efficiency and size of liposome on the oral bioavailability of griseofulvin-loaded liposomes. Pharmaceutics 8 (3), 25 (2016).
11. Naito Y, Ui-Tei K. siRNA design software for a target gene-specific RNA interference. Front. Genet. 3, 102 (2012).
12. Kagami H, Agata H, Tojo A. Bone marrow stromal cells (bone marrow-derived multipotent mesenchymal stromal cells) for bone tissue engineering: basic science to clinical translation. Int. J. Biochem. Cell Biol. 43 (3), 286-289 (2011).
13. Delorme B, Charbord P. Culture and characterization of human bone marrow mesenchymal stem cells. Methods Mol. Med. 140, 67-81 (2007).
14. Lee DE, Koo H, Sun IC, Ryu JH, Kim K, Kwon IC. Multifunctional nanoparticles for multimodal imaging and theragnosis. Chem. Soc. Rev. 41 (7), 2656-2672 (2012).
15. Wang J, Li Y, Nie G. Multifunctional biomolecule nanostructures for cancer therapy. Nat. Rev. Mater. 6 (9), 766-783 (2021).
16. Jiang C, Qi Z, He W et al. Dynamically enhancing plaque targeting via a positive feedback loop using multifunctional biomimetic nanoparticles for plaque regression. J. Control. Release 308, 71-85 (2019).
17. He C, Lu J, Lin W. Hybrid nanoparticles for combination therapy of cancer. J. Control. Release 219, 224-236 (2015).
18. Feng J, Yu W, Xu Z, Hu J, Liu J, Wang F. Multifunctional siRNA-laden hybrid nanoplatform for noninvasive PA/IR dual-modal imaging-guided enhanced photogenetherapy. ACS Appl. Mater. Interfaces 12 (20), 22613-22623 (2020).
19. Habibi N, Quevedo DF, Gregory JV, Lahann J. Emerging methods in therapeutics using multifunctional nanoparticles. Wiley Interdiscip. Rev. Nanomed. Nanobiotechnol. 12 (4), e1625 (2020).
20. Van Tran V, Moon JY, Lee YC. Liposomes for delivery of antioxidants in cosmeceuticals: challenges and development strategies. J. Control. Release 300, 114-140 (2019).
21. Delorme B, Charbord P. Culture and characterization of human bone marrow mesenchymal stem cells. In: Tissue Engineering. Methods in Molecular Medicine. Hauser H, Fussengger M (Eds). Humana Press, NJ, USA (2007).
22. Song S, Sanchez-Ramos J. Preparation of neural progenitors from bone marrow and umbilical cord blood. Methods Mol. Biol. 438, 123-134 (2008).
23. Baghdan E, Pinnapireddy SR, Strehlow B, Engelhardt KH, Scha fer J, Bakowsky U. Lipid coated chitosan-DNA nanoparticles for enhanced gene delivery. Int. J. Pharm. 535 (1-2), 473-479 (2018).
24. Chernikov IV, Vlassov VV, Chernolovskaya EL. Current development of siRNA bioconjugates: from research to the clinic. Front. Pharmacol. 10, 444 (2019).
25. Ui-Tei K, Naito Y, Nishi K, Juni A, Saigo K. Thermodynamic stability and Watson-Crick base pairing in the seed duplex are major determinants of the efficiency of the siRNA-based off-target effect. Nucleic Acids Res. 36 (22), 7100-7109 (2008).
26. Gredell JA, Berger AK, Walton SP. Impact of target mRNA structure on siRNA silencing efficiency: a large-scale study. Biotechnol. Bioeng. 100 (4), 744-755 (2008).
27. Ge Q, Ilves H, Dallas A et al. Minimal-length short hairpin RNAs: the relationship of structure and RNAi activity. RNA 16 (1), 106-117 (2010).
28. Kamola PJ, Nakano Y, Takahashi T, Wilson PA, Ui-Tei K. The siRNA non-seed region and its target sequences are auxiliary determinants of off-target effects. PLOS Comput. Biol. 11 (12), e1004656 (2015).
29. Matranga C, Tomari Y, Shin C, Bartel DP, Zamore PD. Passenger-strand cleavage facilitates assembly of siRNA into Ago2-containing RNAi enzyme complexes. Cell 123 (4), 607-620 (2005).
30. Das S, Ghosal S, Chakrabarti J, Kozak K. SeedSeq: off-target transcriptome database. Biomed. Res. Int. 2013, 905429 (2013).
31. Ozcan G, Ozpolat B, Coleman RL, Sood AK, Lopez-Berestein G. Preclinical and clinical development of siRNA-based therapeutics. Adv. Drug Deliv. Rev. 87, 108-119 (2015).
32. Behlke MA. Chemical modification of siRNAs for in vivo use. Oligonucleotides 18 (4), 305-319 (2008).
33. Wang Y, Xiao H, Fang J et al. Construction of negatively charged and environment-sensitive nanomedicine for tumor-targeted efficient siRNA delivery. Chem. Commun. (Camb.) 52 (6), 1194-1197 (2016).
34. Balazs DA, Godbey W. Liposomes for use in gene delivery. J. Drug Deliv. 2011, 326497 (2011).
35. Chen J, Guo Z, Tian H, Chen X. Production and clinical development of nanoparticles for gene delivery. Mol. Ther. Methods Clin. Dev. 3, 16023 (2016).
36. Pinto-Alphandary H, Andremont A, Couvreur P. Targeted delivery of antibiotics using liposomes and nanoparticles: research and applications. Int. J. Antimicrob. Agents 13 (3), 155-168 (2000).
37. Mufamadi MS, Pillay V, Choonara YE et al. A review on composite liposomal technologies for specialized drug delivery. J. Drug Deliv. 2011, 939851 (2011).
38. Rawicz W, Olbrich KC, McIntosh T, Needham D, Evans E. Effect of chain length and unsaturation on elasticity of lipid bilayers. Biophys. J. 79 (1), 328-339 (2000).
39. Bhattacharya S, Haldar S. Interactions between cholesterol and lipids in bilayer membranes. Role of lipid headgroup and hydrocarbon chain-backbone linkage. Biochim. Biophys. Acta 1467 (1), 39-53 (2000).
40. Needham D, Nunn RS. Elastic deformation and failure of lipid bilayer membranes containing cholesterol. Biophys. J. 58 (4), 997-1009 (1990).
41. Sagredo O, Pazos MR, Satta V, Ramos JA, Pertwee RG, Fernandez-Ruiz J.

Neuroprotective effects of phytocannabinoid-based medicines in experimental models of Huntington's disease. J. Neurosci. Res. 89 (9), 1509-1518 (2011). Provides preclinical evidence in support of a beneficial effect of cannabis-based medicine as a neuroprotective agent capable of delaying disease progression in Huntington's disease (HD).
42. Tubaro A, Giangaspero A, Sosa S et al. Comparative topical anti-inflammatory activity of cannabinoids and cannabivarins. Fitoterapia. 81 (7), 816-819 (2010).
43. Hadinoto K, Sundaresan A, Cheow WS. Lipid-polymer hybrid nanoparticles as a new generation therapeutic delivery platform: a review. Eur. J. Pharm. Biopharm. 85 (3 Pt A), 427-443 (2013).
44. Iuvone T, Esposito G, De Filippis D, Scuderi C, Steardo L. Cannabidiol: a promising drug for neurodegenerative disorders? CNS Neurosci. Ther. 15 (1), 65-75 (2009).
45. Touitou E, Illum L. Nasal drug delivery. Drug Deliv. Transl. Res. 3 (1), 1-3 (2013).
46. Nagarkatti P, Pandey R, Rieder SA, Hegde VL, Nagarkatti M. Cannabinoids as novel anti-inflammatory drugs. Future Med. Chem. 1 (7), 1333-1349 (2009). Focuses on the potential use of cannabinoids as anti-inflammatory agents in inflammatory and autoimmune diseases triggered by activated T cells and other cellular immune components.

TABLE 1
Sequences.

| SEQ | | |
|---|---|---|
| ID NO | Description | Sequence |
| 1 | E30 dsRNA without overhangs | CUGCUUUAGUCGAGAACCA |
| 2 | E30 siRNA strand 1 | CUGCUUUAGUCGAGAACCAuu |
| 3 | E30 siRNA strand 2 | UGGUUCUCGACUAAAGCAGga |
| 4 | dsRNA with overhangs sense | GAAUCGAGAUCGGAUGUCAtt |
| | strand | |
| 5 | dsRNA with overhangs antisense | UGACAUCCGAUCUCGAUUCag |
| | strand | |

Lower case letter indicates unpaired nucleotide in dsRNA, i.e., overhang nucleotides.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references may be made herein. The cited references are incorporated by reference herein in their entireties. In the event that there is an inconsistency between a definition of a term in the specification as compared to a definition of the term in a cited reference, the term should be interpreted based on the definition in the specification.

1. A nanoparticle comprising:
(i) a core comprising at least one siRNA; and
(ii) a phospholipid outer layer.

2. The nanoparticle of claim 1, wherein the core further comprises chitosan or chitosan lactate.

3. The nanoparticle of claim 1, wherein the phospholipid outer layer comprises at least one anti-inflammatory compound.

4. The nanoparticle of claim 3, wherein the at least one anti-inflammatory compound comprises cannabidiol (CBD).

5. The nanoparticle of claim 1, wherein the at least one siRNA comprises SEQ ID NO:
1.

6. The nanoparticle of claim 1, wherein the phospholipid outer layer comprises dipalmitoylphosphatidylcholine (DPPC), 1,2-dioleoyl-sn-glycero-3-phosphocholine (DOPC), or both DPPC and DOPC.

7. A nanoparticle comprising:
(i) a core comprising at least one siRNA wherein the at least one siRNA comprises SEQ ID NO: 1, or an siRNA at least 90% similar to SEQ ID NO: 1 and the core further comprises chitosan lactate;
(ii) a phospholipid outer layer comprising DPPC, DOPC, or both DPPC and DOPC and the phospholipid outer layer further comprising cannabidiol (CBD).

8. The nanoparticle of claim 7, wherein the phospholipid outer layer comprises DPPC, DOPC, and cannabidiol wherein the final ratio of chitosan lactate is CSL:DPPC:DOPC:CBD is about 20:12:10.5:7.5:1.

9. The nanoparticle of claim 7, wherein the ξ-potential of the nanoparticle is about 6 mV to about 55 mV.

10. The nanoparticle of claim 7, wherein the ξ-potential of the nanoparticle is about 6 mV to about 8 mV.

11. The nanoparticle of claim 7, wherein the nanoparticle is about 150 to about 210 nm in diameter.

12. The nanoparticle of claim 11, wherein the nanoparticle is about 195 to about 207 nm in diameter.

13. An siRNA where the siRNA comprises SEQ ID NO: 1, or an siRNA at least 90% similar to SEQ ID NO: 1.

14. The siRNA of claim 13, wherein the at least one modified nucleotide comprises a O-methylated nucleotide or a phosphorothioate-modified nucleotide.

15. A pharmaceutical composition comprising the nanoparticle of claim 7.

16. A pharmaceutical composition comprising the siRNA of claim 13.

17. A method of treating a neurological disease or disorder in a subject in need thereof, the method comprising administering a therapeutically effective amount of the pharmaceutical composition of claim 15 to a subject to treat the neurological disease or disorder, wherein the neurological disease or disorder is a neurodegenerative disease selected from the group consisting of Huntington's disease, familial Parkinson's disease, familial Alzheimer's disease, and familial amyotrophic lateral sclerosis.

18. The method of claim 17, wherein the neurodegenerative disease is Huntington's disease.

19. The method of claim 17, wherein administration comprises intranasal administration of the pharmaceutical composition.

20. A method of making the nanoparticle of claim 7, comprising
(i) preparing a nanoparticle comprising:
a core comprising chitosan lactate and a phospholipid outer layer comprising DPPC, DOPC, or both DPPC and DOPC and the phospholipid outer layer further comprising cannabidiol (CBD);
(ii) loading the core with the siRNA of claim 13.


SEQUENCE LISTING (5 sequences)

SEQ ID NO: 1 - RNA, 19 nt, synthetic construct
  source 1..19: mol_type = other RNA
ctgctttagtcgagaacca

SEQ ID NO: 2 - RNA, 21 nt, synthetic construct
  source 1..21: mol_type = other RNA
ctgctttagtcgagaaccatt

SEQ ID NO: 3 - RNA, 21 nt, synthetic construct
  source 1..21: mol_type = other RNA
tggttctcgactaaagcagga

SEQ ID NO: 4 - DNA, 21 bp, synthetic construct
  source 1..21: mol_type = other DNA
  misc_feature 1..19: note = RNA
  misc_feature 20..21: note = DNA
gaatcgagatcggatgtcatt

SEQ ID NO: 5 - RNA, 21 nt, synthetic construct
  source 1..21: mol_type = other RNA
tgacatccgatctcgattcag